(19) United States
(12) Patent Application Publication
(10) Pub. No.: US 2023/0178232 A1
(43) Pub. Date: Jun. 8, 2023

(54) Template and Method for Inputting and Disseminating Occupational and Physical Therapy Information

(71) Applicant: Prachi Rathi, Jacksonville, FL (US)

(72) Inventor: Prachi Rathi, Jacksonville, FL (US)

(21) Appl. No.: 17/545,114

(22) Filed: Dec. 8, 2021

(51) Int. Cl. G16H 40/67; G16H 20/30; G16H 50/20

(52) U.S. Cl. CPC G16H 40/67; G16H 20/30; G16H 50/20

(57) ABSTRACT

Through the use of a computer-based or mobile-based web application, an occupational therapist can enter data into the fields of a template that have been established for that purpose. The specific fields may depend on a particular situation or the particular needs of an individual and this program allows the occupational therapist/the assessor to customize the needs to fit the individual circumstances of the person. Certain presets have been included in the system that will alert the occupational therapist/the assessor to the problem areas, as well as an array of possible solutions as well as a ranking of priority of possible solutions.

### FIELD OF THE INVENTION

This relates to the field of occupational therapy and specifically needs of individuals who have been released from surgical centers or hospitals or ambulatory centers after a medical condition or weakness related to any physical/physiological ill-health that may or may not require the use of certain assistive devices to aid in their recovery.

It is important to appropriately assess whether an individual needs home health aides and/or environmental modifications within the home so that they can live independently and safely as quickly as and as long as possible.

### PRIOR ART

There are many prior art references to devices and methods to provide the actual equipment as well as various types of devices that are used in the home. Such items may include elongated implements, reachers/grabbers, and the like. The specific types of accommodations or aids depends entirely on the needs of the individual. This application is not directed specifically at those pieces of equipment but is instead an assessment tool that is web based to be used by an occupational therapist and/or a physical therapist or other qualified professional to determine the need(s) of an individual in his or her own home as he or she recovers from a medical condition or a family must adapt their home to ensure comfort of their loved ones with special needs. None of the prior art teach a computer based application that can be accessed by multiple individuals and includes all the features that are taught in this application including the ability to perform assessments remotely with the use of photographs or pictures, live or stored video or audio Speech to Text.

### BRIEF SUMMARY OF THE INVENTION

When an individual has undergone a serious medical problem and this may include a stroke, a heart attack, (generally speaking cardio-pulmonary or neuro-muscular limitations or) an operation, surgery or has sustained injuries from an automobile accident or industrial injury, it is not uncommon for the individual to need certain assistive devices when the person is discharged from the hospital or rehabilitation or long term facility to the person's home either on a short term or long term basis. The role of the occupational therapist is to evaluate the home and surroundings and recommend environmental modification as well as assistive devices to aid the person during his or her recovery process and safe living at the person's home. There is value this application for people who are choosing to Age-in-Place and thus adapt the home to their current as well as upcoming needs considering the aging process. Although much of this application will focus on the role of an occupational therapist in a person's recovery, this application can also be used by other trained medical personnel such as a physical therapist or nurse or Case manager or family member could also use this application (in a limited capacity i.e. data collection) and recommendations would be made by a qualified therapist/professional.

It is the goal when someone is being discharged to their home to provide a safe environment so that the person can live and function independently and the family members can be reassured that all appropriate environmental modifications in the home have been considered and completed according to the family's current time and financial capabilities to ensure the person's optimal safety.

This device is not directed to the actual assistive devices but is instead directed to a method whereby an occupational therapist can either enter a home and make appropriate recommendations to the homeowner in terms of potential environmental modifications to the home (incorporating Energy Conservation and Work Simplification suggestions) or perform this analysis remotely either through pictures or video that can be uploaded by the assessor and downloaded or viewed on the web-portal by the therapist for analysis if needed. This is done on a computer-based web platform or mobile application where the occupational therapist can specifically delineate certain needs of an individual and address those needs in the template. The information on the template can be transmitted to the appropriate individual and/or any medical providers. Certain fields in the template may include needs for lifts such as a chair lift or assistive devices that would enable an individual to move up and down stairs or grabbers that can enable a person to retrieve items from shelves that may become inaccessible due to the medical condition.

The current application has three particular features that can assist both the occupational therapist, the caregiver, the patient and the medical provider(s): a plurality of events or occurrences where the occupational therapist is directed to a potential problem (“red flags”), a library of possible solutions that have been stored in the program (“knowledge bank”) and a way to prioritize or utilize the resources of the individual as effectively as possible based on the person's physical, psycho-social and financial state (“priority features”). The presets that alert the occupational therapist to a problem may be changed by the therapist depending on the nature of the case but there will be general presets that have been predetermined as part of the software.

### BRIEF DESCRIPTION OF THE DRAWINGS

There are no drawings required.

### DETAILED DESCRIPTION OF THE EMBODIMENTS

In this application the occupational therapist will use a computer, either a desktop computer or a portable electronic device such as a laptop, or handheld electronic modalities (for e.g. tablets, phones, etc., to store or access certain necessary information regarding an individual such as name, address, the nature of the illness or disability, specific assistive device that may be prescribed or recommended by the health care provider, and other pertinent medical information. The template that is used in this application is designed specifically for an occupational therapist (or other qualified professional) to evaluate and assess the needs of a patient as it relates to a person (usually a loved one) when they are discharged from the hospital or rehabilitation facility or after an injury or illness to their home. It can be used also in some cases for people who are preferring to Age-in-Place.

To ensure that the needs of the individual are appropriately addressed, the needs of the individual who is undergoing recovery are addressed by an occupational therapist (or other qualified professional) who might visit the home in person or may assess or evaluate the data entered in the program along with pictures and videos by other entities (such as Home Care Aides, Family members or friends, other health care professionals) and who makes appropriate recommendations. These recommendations may include a variety of accommodations to the home depending on the needs of the individual. Some examples may include modifying a kitchen or bathroom space to make the area more easily accessible for the wheelchair bound patient, for example. Other accommodations may include the placement of handrails in the tub or shower area for persons who may have mobility issues. The advice of a skilled occupational therapist (or other qualified and trained professional) is necessary to fully evaluate the home to assess the particular needs of the patient and be able to communicate those recommendations to the loved one.

Additionally, an occupational therapist (or other qualified and trained (professional) may be able to make the appropriate evaluation and needed evaluation remotely with modern technology using this template including future upgrades for e.g. incorporating additional data fields and technology solutions to ensure ease of use of the software. There may be a potential for research of a variety of home safety related topics.

For example, the caregiver of the person or the individual themselves may take pictures of the living space either in the form of still pictures or video and upload/download these images into the application to assist a qualified and trained professional like an occupational therapist for inclusion in the template. Once the occupational therapist has received the visual images, the images may be stored electronically for later use. The ability to review these images remotely enable the occupational therapist to become more productive. An added benefit is the ability to store the images so that multiple skilled professionals can review the environment in the event that additional modifications may become necessary; with modern technology and the use of this template these recommendations can be made without the need for an in-person home visit.

In the typical case an occupational therapist will evaluate the home by traveling to the home to make the assessment. This requires the coordination of the occupational therapist as well as the person who is in recovery and the caregiver of the person. With the ability to remotely evaluate a living area using the ability to upload/download pictures and/or video the occupational therapist's time is much better utilized and not dependent entirely on the schedule of third parties.

This evaluation can be done either in real time (synchronous) with video capability so that the occupational therapist can discuss the situation directly and in person through a computer screen with the homeowner or caregiver in real time or at different times (asynchronous). Regardless of the timing of the evaluation the caregiver will be provided access to the information.

All the inputted information will be stored on a computer/web based template which may have a variety and multitude of fields to be completed by an occupational therapist depending on the particular need(s) of the individual. The information will have sufficient security means to enable only certain individuals to access the pertinent information. Additionally, the occupational therapist can have access to the notes and be able to change their recommendations or at least indicate what recommendations have been accepted by the individual or homeowner or a family member so that progress can be documented by the occupational therapist or any qualified and trained professional.

The use of a template will compartmentalize the information and allow the information to be easily transmitted from care provider to the therapist or therapist to therapist or from occupational therapist to medical provider or payer or to the patient or the client's loved one.

The current application has three particular features that can assist all users (the occupational therapist, the caregiver, the patient and the medical provider(s)): a plurality of events or occurrences where the occupational therapist is directed to a potential problem (“red flags”), a library of possible solutions that have been stored in the program (“knowledge bank”) and a way to prioritize solutions or utilize the resources of the individual as effectively as possible based on the person's physical, psycho-social and financial state (“priority features”). The presets that alert the occupational therapist to a problem may be changed by the therapist depending on the nature of the case but there will be general presets that have been inputted into the system.

The first of these three features (“red flags”) is a plurality of predetermined factors that will alert the occupational therapist (or other qualified professional) to take a remedial action. As the occupational therapist inserts certain information into the template the program identifies a problem based on presets that have been established with the program. Once a red flag or problem is identified, the therapist is directed to find a resolution of the issue depending on the specific need of the patient by linking to the knowledge base that is discussed in further detail. For instance, if the person has limited mobility and finds it difficult to travel over a threshold the therapist will insert that data into the template and the program will then notify the therapist that the issue needs to be resolved. Another example may be that a person may be wheelchair bound and may find it difficult to reach cabinets or light switches. Once the information is inputted into the program by the therapist, the program will then identify a problem and further notify the therapist that a solution needs to be created at the time. The purpose of this part of the template is to ensure that the person's home has been fully evaluated and any and all possible problems aka “red flags” have been addressed before the person returns home safely or the home is adequately safe for a person to Age-in-Place in an optimally safe environment.

An additional unique feature of the program is the ability to store certain possible solutions to any possible problem areas by accessing a database (knowledge bank) where possible solutions have been stored and information is added as new solutions are creatively discovered by a variety of professionals. This database of possible solutions will also depend on the location of the property and any applicable building codes for the area so that the appropriate solution may be achieved. The portion of the program that identifies the problem (red flag) is linked to the database (knowledge bank) so that an appropriate solution can be proposed. For instance, in the example of the person who is wheelchair bound that person may find it difficult to access shelves the knowledge base may suggest a reaching device or a grabber to assist the person.

A third area that is important for anyone caring for someone in the home as well as the patient is the appropriate allocation of resources. Once the information is placed into the program by the occupational therapist and a red flag is identified the knowledge bank is opened for possible solutions. Once the possible solutions have been identified the program in conjunction with the occupational therapist/qualified professional will suggest solutions based on the priority level of the “red flag” based on the person's physical, psycho-social and financial state. In the example of the person who is wheelchair bound, the purchase of a grabber to reach items is a relatively minor expense as opposed to a reconfiguration of the kitchen area such as lowering cabinets, which would be much more costly. Contained within this section of the program will be reminders that will alert to occupational therapist/the evaluating professional to reassess the situation and the possible solutions including an estimation of the cost involved.

This device will be used to use software and a template to input information regarding a patient who needs medical services, whether occupational services or physical therapy services. This is a web-based application.

The key players in this application will include the patient, occupational and/or physical therapist, a case manager and the patient's doctor. The information that can be inputted into the template may be made by the patient, occupational therapist or nurse or case manager. The medical doctor can provide specific medical guidance using this template as well. This guidance from the medical doctor will be viewable by the patient, occupational therapist or nurse case manager or whoever is eligible to access this data.

With this device the occupational therapists and/or physical therapists or other qualified medical personnel can enter assessments or analyses based on data that has been uploaded to the program. The judgment of the qualified medical person would initiate these assessments and analysis.

The user, such as a loved one of the individual who is having medical difficulties, can provide data or information into the program to be forwarded to the occupational therapist or other qualified medical professional. The user can put notes and then upload the information to the program to the template. This information may also include a picture or a short video of the living space of the loved one.

The patient's doctor can also upload information concerning the diagnosis and/or prognosis of the patient and can also upload any possible recommended treatment for the patient. Appropriate HIPPA laws concerning disclosure of any medical information of the patient would be honored. The doctor cannot make any assessments or analysis in this program. These assessments or analysis are restricted to the occupational therapist, physical therapist or other qualified medical provider based on a consideration of the totality of factors.

The occupational therapist inputs information into a template including recommendations concerning specific items that may assist the patient in his or her recovery and/or long-term residing in a particular location. The occupational therapist can then prioritize those items for the patient's convenience and the patient's needs.

The information can be inputted using a portable electronic device and will be based in a mobile application (hereafter “the app”), and it can be used, and it can be accessed using any mobile or portable electronic device.

The occupational therapist can use the app on a mobile device or it can be accessed through the software.

In addition, video and audio (Speech to Text) components will also be available for ease of use for the patient and/or occupational therapist. All information will be stored in the cloud and it can be archived by the participants.

The information can be accessed in real time as well as Store-Forward, which is a technical term used in its field. The applicant will now describe the use of the device in two examples.

Example 1: In the first example, assume that the patient has had a hip injury. Depending on the type of hip injury, there is likely a predictable period of recovery.

Occupational and/or physical therapy services will likely be required to rehabilitate that individual safely within the person's home.

The necessary medical information is provided by the medical doctor so that the occupational therapist/the assessor has a general idea of the nature of the patient's medical condition.

The occupational therapist/the assessor may suggest or recommend to the patient to take a video of a person's home and upload to the app so that the occupational therapist can make specific recommendations based on the layout of the home. Certain recommendations can include grabbers or hand bars for showers for instance with the individual who has a hip injury and may find it difficult to walk or bend over.

The patient would upload the video into the template and the occupational therapist/the assessor would analyze the video and make specific recommendations regarding the needs of the patient. The occupational therapist would also prioritize those recommendations.

In the first example, if the person has difficulty with his or her hands, an audio (Speech to Text) component is incorporated into the device so that the individual can simply dictate into the app.

In the instance of the individual with a hip fracture, there may be a need for a grab handle in the shower. Occupational therapist/the assessor would request that the person take a video and this video is uploaded to the app for analysis.

In the example of a grab bar or handle, the occupational Therapist/the assessor can then recommend a specific type of handle based on the specific needs of the individual. There will also be a link built into the app to certain specified manufacturers for grab bars/handles or other related equipment or solutions for bathrooms or the home as recommended by the occupational therapist to make it easier for the therapist as well as the end user.

Example 2: In this second example, the individual is in an retirement living facility or at home with the same hip fracture. In this case, a nurse or case manager or care provider in the facility/home will enter the individual's living areas and take a video of the room(s) on behalf of the patient and upload that information to the app, which is part of this device.

The occupational therapist would then review the information that was provided by the nurse case manager, and again make specific recommendations concerning the needs of a particular patient. Both the patient and/or nurse case manager (or relevant care providers) will have access to the information that is contained in the application based upon predetermined qualifications and determinations by a particular organization's policies or client's personal preferences re: sharing of their Personal Health Information (PHI)/data.

All the information that is inputted to the device can be archived to the cloud. In this matter, this application would make the process more efficient because the occupational therapist is not required to travel to the person's home to make specific assessments but instead could have a visual reference by downloading a picture of the person's home. It would also assist the patient, particularly those who have difficulty ambulating, in that it makes it more convenient for them to provide information for the occupational therapist without the need to travel.

It is important to note that each situation is different and that individual needs will vary depending on the particular application. The advice and counsel of a qualified occupational therapist and/or physical therapist is absolutely essential in this process both for gathering the information as well as updating the information.

The program would also provide links to providers of durable medical equipment or building contractors or home remodelers depending on the needs of the individual.

The inventor claims:

1. A template on a web-based platform for inputting and disseminating occupational and physical therapy information which is comprised of
web-based software;
a portable electronic device;
a template;
wherein the template is on the web-based platform;
wherein a certain designated number of fields are provided on the template;
wherein information is inserted into the fields on the template;
a qualified medical provider;
wherein the qualified medical professional inputs assessments into the templates based on analysis of the situation;
end-user;
wherein the end-user can upload data to the template;
said data that is provided by the end user is accessed by the qualified medical professional;
wherein the qualified medical professional medical can provide prognosis;
wherein predetermined red flags are provided;
wherein the red flags provide notifications based on preset information in the program;
wherein a knowledge bank is provided;
said knowledge bank provide possible solutions for a red flag;
wherein presets determine priority of action steps for a red flag;
wherein access to the information may be restricted.

2. A template on a web-based platform for inputting and disseminating occupational and physical therapy information as described in claim 1 wherein the portable electronic device is a cell phone.

3. A template on a web-based platform for inputting and disseminating occupational and physical therapy information as described in claim 1 wherein the portable electronic device is a tablet.

4. A template on a web-based platform for inputting and disseminating occupational and physical therapy information as described in claim 1 wherein information can be uploaded using video capability.

5. A template on a web-based platform for inputting and disseminating occupational and physical therapy information as described in claim 1 wherein information can be uploaded using audio Speech to Text capability.

6. A template on a web-based platform for inputting and disseminating occupational and/or physical therapy information as described in claim 1 wherein links may be provided to providers of durable/home medical equipment as well as various contractors/home remodelers and other related healthcare service providers' information.

7. A template on a web-based platform for inputting and disseminating occupational and/or physical therapy information as described in claim 1 wherein recommendations can be prioritized.
